(19) United States
(12) Patent Application Publication
(10) Pub. No.: US 2022/0184133 A1
(43) Pub. Date: Jun. 16, 2022

(54) USE OF MESENCHYMAL STEM CELLS IN PREPARATION OF FORMULATION FOR PROMOTING FAT TRANSPLANTATION

(71) Applicant: UNIVERSITY OF MACAU, Macau (CN)

(72) Inventors: Renhe XU, Macau (CN); Roma Pradeep BORKAR, Macau (CN); Dejin ZHENG, Macau (CN); Enqin LI, Macau (CN)

(73) Assignee: UNIVERSITY OF MACAU, Macau (CN)

(21) Appl. No.: 17/258,726

(22) PCT Filed: Jul. 17, 2020
(86) PCT No.: PCT/CN2020/102710

§ 371 Date: Jan. 7, 2021

(30) Foreign Application Priority Data
May 28, 2020 (CN) 202010470733.7

(51) Int. Cl. A61K 35/28; C12N 5/0775; A61P 17/02

(52) U.S. Cl. CPC A61K 35/28; C12N 2506/02; A61P 17/02; C12N 5/0662

(57) ABSTRACT

The present disclosure discloses use of mesenchymal stem cells in preparation of a formulation for promoting fat transplantation, relating to the field of biotechnologies. The inventors found through research that compared with MSCs derived from somatic cells, MSCs derived from human pluripotent stem cells have more stable quality, are not affected by donor's physical quality, disease and treatment process, and can promote fat transplantation by enhancing tissue remodeling, angiogenesis and adipose cell survival and decreasing tissue fibrosis.

### CROSS-REFERENCE TO RELATED APPLICATION

The present disclosure claims priority of Chinese patent application with the filing number 202010470733.7 filed on May 28, 2020 with the Chinese Patent Office, and entitled “Use of Mesenchymal Stem Cells in Preparation of Formulation for Promoting Fat Transplantation”, the contents of which are incorporated herein by reference in entirety.

### TECHNICAL FIELD

The present disclosure relates to the field of biotechnologies, and in particular to use of mesenchymal stem cells in preparation of a formulation for promoting (i.e., improving) fat transplantation.

### BACKGROUND ART

Autologous fat augmentation has been used for over 100 years to correct and repair soft tissue malformation. It is mainly used for remodeling of face and other parts of body after cancer surgery, scar and wrinkle repair and breast augmentation. Autologous fat is usually extracted from subcutaneous adipose tissues, and has become the most commonly used filling material due to its advantages of natural morphology, high biocompatibility, low cost, non-immunogenicity and no contamination by foreign pathogens.

Nevertheless, the survival rate of grafts is very unstable (25-90%). Besides, autologous fat transplantation still has many obstacles due to partial necrosis caused by lack of angiogenesis. The survival of fat grafts relies on rapid formation of blood vessels so as to provide oxygen and nutrients. However, insufficient blood supply may result in death of a large number of adipose cells, especially for cells in the center of grafts, which in turn easily causes fibrosis and even graft shrinkage.

### SUMMARY

The present disclosure provides use of mesenchymal stem cells in preparation of a formulation for promoting fat transplantation, and a formulation, wherein the mesenchymal stem cells are derived from human pluripotent stem cells including human embryonic stem cells and human induced pluripotent stem cells.

One or more embodiments of the present disclosure provide use of mesenchymal stem cells in preparation of a formulation for promoting fat transplantation, wherein the mesenchymal stem cells are derived from human pluripotent stem cells.

One or more embodiments of the present disclosure provide use of mesenchymal stem cells in promoting fat transplantation, wherein the mesenchymal stem cells are derived from human pluripotent stem cells.

One or more embodiments of the present disclosure provide a method for promoting fat transplantation, including applying mesenchymal stem cells to a subject in need thereof, wherein the mesenchymal stem cells are derived from human pluripotent stem cells.

One or more embodiments of the present disclosure provide use of mesenchymal stem cells in preparation of a formulation for activating a CCL2 signal pathway in early stage of fat transplantation, wherein the mesenchymal stem cells are derived from human pluripotent stem cells.

One or more embodiments of the present disclosure provide use of mesenchymal stem cells in activating a CCL2 signal pathway in early stage of fat transplantation, wherein the mesenchymal stem cells are derived from human pluripotent stem cells.

One or more embodiments of the present disclosure provide a method for activating a CCL2 signal pathway in early stage of fat transplantation, including applying mesenchymal stem cells to a subject in need thereof, wherein the mesenchymal stem cells are derived from human pluripotent stem cells.

One or more embodiments of the present disclosure provide use of mesenchymal stem cells in preparation of a formulation for gathering macrophages in early stage of fat transplantation, wherein the mesenchymal stem cells are derived from human pluripotent stem cells.

One or more embodiments of the present disclosure provide use of mesenchymal stem cells in gathering macrophages in early stage of fat transplantation, wherein the mesenchymal stem cells are derived from human pluripotent stem cells.

One or more embodiments of the present disclosure provide a method for gathering macrophages in early stage of fat transplantation, including applying mesenchymal stem cells to a subject in need thereof, wherein the mesenchymal stem cells are derived from human pluripotent stem cells.

One or more embodiments of the present disclosure provide use of mesenchymal stem cells in preparation of a medicine for promoting regathering of adipose cells, wherein the mesenchymal stem cells are derived from human pluripotent stem cells.

One or more embodiments of the present disclosure provide use of mesenchymal stem cells in promoting regathering of adipose cells, wherein the mesenchymal stem cells are derived from human pluripotent stem cells.

One or more embodiments of the present disclosure provide a method for promoting regathering of adipose cells, including applying mesenchymal stem cells to a subject in need thereof, wherein the mesenchymal stem cells are derived from human pluripotent stem cells.

One or more embodiments of the present disclosure provide use of mesenchymal stem cells in preparation of a medicine for promoting revascularization, wherein the mesenchymal stem cells are derived from human pluripotent stem cells.

One or more embodiments of the present disclosure provide use of mesenchymal stem cells in promoting revascularization, wherein the mesenchymal stem cells are derived from human pluripotent stem cells.

One or more embodiments of the present disclosure provide a method for promoting revascularization, including applying mesenchymal stem cells to a subject in need thereof, wherein the mesenchymal stem cells are derived from human pluripotent stem cells.

One or more embodiments of the present disclosure provide use of mesenchymal stem cells in preparation of a medicine for improving survival rate of adipose cells, wherein the mesenchymal stem cells are derived from human pluripotent stem cells.

One or more embodiments of the present disclosure provide use of mesenchymal stem cells in improving survival rate of adipose cells, wherein the mesenchymal stem cells are derived from human pluripotent stem cells.

One or more embodiments of the present disclosure provide a method for improving survival rate of adipose cells, including applying mesenchymal stem cells to a subject in need thereof, wherein the mesenchymal stem cells are derived from human pluripotent stem cells.

One or more embodiments of the present disclosure provide use of mesenchymal stem cells in inhibiting tissue fibrosis, wherein the mesenchymal stem cells are derived from human pluripotent stem cells.

One or more embodiments of the present disclosure provide a method for inhibiting tissue fibrosis, including applying mesenchymal stem cells to a subject in need thereof, wherein the mesenchymal stem cells are derived from human pluripotent stem cells.

The present disclosure provides a method for promoting fat transplantation, comprising applying mesenchymal stem cells to a subject in need thereof, wherein the mesenchymal stem cells are derived from human pluripotent stem cells including human embryonic stem cells and induced pluripotent stem cells.

In one or more embodiment, the method comprises activating a CCL2 signal pathway in an early stage of fat transplantation, comprising applying the mesenchymal stem cells to a subject in need thereof.

In one or more embodiment, the method comprises gathering macrophages in an early stage of fat transplantation, comprising applying the mesenchymal stem cells to a subject in need thereof.

In one or more embodiment, the method comprises promoting regathering of adipose cells, comprising applying mesenchymal stem cells to a subject in need thereof.

In one or more embodiment, the method comprises promoting revascularization, comprising applying mesenchymal stem cells to a subject in need thereof.

In one or more embodiment, the method comprises improving survival rate of adipose cells, comprising applying mesenchymal stem cells to a subject in need thereof.

In one or more embodiment, the method comprises inhibiting tissue fibrosis, comprising applying mesenchymal stem cells to a subject in need thereof.

In one or more embodiment, a method for preparing the mesenchymal stem cells comprises: differentiating the human pluripotent stem cells into the mesenchymal stem cells, culturing differentiated mesenchymal stem cells, and subculturing once every 5˜7 days.

The Beneficial Effects at Least Include:

the examples of the present disclosure provide the use of mesenchymal stem cells, derived from human embryonic stem cells (abbreviated as EMSCs), in preparation of a formulation for promoting fat transplantation. Research finds that compared with to MSCs derived from somatic cells, EMSCs have more stable quality, are not affected by donor's physical quality, disease and treatment process, and can promote fat transplantation by enhancing tissue remodeling, angiogenesis and adipose cell survival and decreasing tissue fibrosis.

### BRIEF DESCRIPTION OF DRAWINGS

In order to more clearly illustrate technical solutions of examples of the present disclosure, accompanying drawings which need to be used in the examples will be introduced below briefly. It should be understood that the accompanying drawings below merely show some examples of the present disclosure, therefore, they should not be considered as limitation to the scope, and a person ordinarily skilled in the art still could obtain other relevant accompanying drawings according to these accompanying drawings, without any inventive effort.

FIG. 1 shows graphs of results of EMSCs promoting regathering and transplantation of human fat after extraction provided in an Example and Test Example 1 of the present disclosure;

FIG. 2 shows graphs of results of EMSCs promoting fat regathering and retention provided in Test Example 1 of the present disclosure;

FIG. 3 shows graphs of experimental results of retention and differentiation of EMSCs in fat grafts in Test Example 2 of the present disclosure;

FIG. 4 shows graphs of results of host mouse tissues participating in transplantation of human fat in Test Example 2 of the present disclosure;

FIG. 5 shows graphs of transcriptomic analysis of fat grafts in Test Example 3 of the present disclosure;

FIG. 6 shows graphs of mouse immune response and expression of inflammation-related genes in EMSC group in Test Example 3 of the present disclosure;

FIG. 7 shows graphs of experimental results of dependence of EMSCs' effect of promoting transplantation on CCL2 in Test Example 4 of the present disclosure;

FIG. 8 shows graphs of experimental results of effect of CCL2 in EMSC promoting fat transplantation in Test Example 4 of the present disclosure;

FIG. 9 shows graphs of experimental results of dependence of EMSCs or PBS group on macrophage for fat transplantation in Test Example 5 of the present disclosure; and

FIG. 10 shows graphs of results of RNA sequencing analysis in Test Example 5 of the present disclosure.

### DETAILED DESCRIPTION OF EMBODIMENTS

In order to make the objects, technical solutions and advantages of the examples of the present disclosure clearer, the technical solutions in the examples of the present disclosure will be described below clearly and completely. If no specific conditions are specified in the examples, they are carried out under normal conditions or conditions recommended by manufacturers. If manufacturers of reagents or apparatuses used are not specified, they are conventional products commercially available.

Use of mesenchymal stem cells in preparation of a formulation for promoting fat transplantation and a formulation in the examples of the present disclosure are specifically described below.

Definitions of Terms

“MSCs” herein refer to mesenchymal stem cells, a kind of multipotent stem cell, a group of cells with multipotency present in mesenchyme of the vast majority of tissues of the body, and participating in tissue homeostasis, modification and regeneration processes. Once isolated from tissues and proliferating in vitro, MSCs are capable of self-renewal, propagation and differentiation into a variety of other types of cells, for example, osteoblasts, chondrocytes, adipose cells, vascular smooth muscles, endothelial cells, nerve cells and hepatocytes. Unlike other types of stem cells, MSCs have remarkable immunomodulatory function.

“hESCs” herein refer to human embryonic stem cells, a class of cells that are derived from early embryos and in an undifferentiated state, can self-differentiate and self-renew in a long term, and have the potential to differentiate under certain conditions to form various histocytes. Under supporting condition with definite ingredients, hESCs substantially can maintain their gene homeostasis.

“EMSCs” herein refer to MSCs derived from hESCs.

As used herein, the term “CCL2”, a member of chemokines, is interchangeably used with “C-C-Motif Ligand (2(C-C Motif) Ligand 2)”, which plays an important role in tissue damage and repair in many inflammatory diseases.

### Technical Solution

Previous researches have shown that, compared with fat extract transplantation alone, using fat extract mixed with Ad-MSCs (fat-derived mesenchymal stem cells) to correct craniofacial microsomia significantly improves the volume and survival rate of adipose cells in grafts. However, MSCs derived from somatic cells are unstable in quality, and easily contaminated by pathogens due to dependence on donor's donation. In addition, the quality of patient-derived Ad-MSCs further may be affected by the disease itself and the treatment process, and it is difficult to obtain large quantities of fat extract from bodies of thin and small patients.

On this basis, an embodiment of the present disclosure provides use of mesenchymal stem cells in preparation of a formulation for promoting fat transplantation, wherein the mesenchymal stem cells are derived from human embryonic stem cells, i.e., differentiated from a hESC cell line.

Previous researches have shown that MSCs derived from somatic cells can promote fat transplantation by: (1) paracrine (major pathway); and (2) direct differentiation into fat and vascular cells (secondary pathway). Nevertheless, it remains unclear what factors mediate their function of promoting transplantation. In the present disclosure, using hESCs to produce MSCs (EMSCs), the inventors found that EMSCs could promote fat transplantation, and revealed a new mechanism of mediating the promoting effect by a method such as live cell tracing, RNA sequencing, and gene knockout.

As can be seen at day 1 after transplantation, EMSCs-containing fat grafts quickly became sticking to each other, and still remained intact after being suspended in PBS, while grafts in a control group were looser, and quickly separated after being suspended in PBS. EMSCs helped adipose tissues stick together like glue by promoting remodeling of extracellular matrix (ECM) and regathering of adipose cells. This is a new finding, and this function helps EMSCs promote fat transplantation. In some embodiments, the mesenchymal stem cells derived from human pluripotent stem cells further promote fat transplantation by improving revascularization and/or survival rate of adipose cells in early stage of transplantation.

It can be seen that at days 14, 30 and 90 after transplantation, the EMSCs-containing grafts had better transplantation effects than that of the control group, reflected in that the grafts were larger and heavier and contained more capillaries on their surfaces. Histological analysis also revealed presence of more adipose cells and vascular cells, and fewer fibrotic and necrotic tissues in the EMSCs-containing grafts. Although most of EMSCs within the grafts disappeared rapidly, a few EMSCs still could persist by day 90 after transplantation, wherein part of the EMSCs were also differentiated into adipose cells and vascular cells.

In order to analyze the molecular mechanism of the above effect of promoting transplantation, the inventors collected fat grafts (with or without EMSCs) from day 1 to day 90 after transplantation and carried out RNA sequencing. Results showed that genes related to VEGF signal pathway, PPAR signal pathway, cytokine interaction and ECM formation were mostly up-regulated in the EMSCs group. Among these genes, CCL2 in the EMSCs group was highly expressed from the first day after transplantation and continued to day 30, although the expression decreased in later stage.

The effect of CCL2 secreted by the EMSCs in fat transplantation was detected by knocking out the CCL2 gene in the EMSCs, and results showed that the promoting effect of the EMSCs, with CCL2 being knocked out, on fat grafts was reduced, reflected in reduction of size, weight, number of blood vessels and quality of the grafts. In-vitro experiments showed that the gathering capability of EMSCs to macrophages declined after CCL2 was knocked out. Thus, the CCL2/Ccr2 signal pathway can help gather host macrophages into the grafts so as to promote fat regeneration.

Macrophages play different roles in different stages of fat transplantation, for example, promoting ECM remodeling and revascularization in the grafts in early stage, while promoting graft fibrosis in later stage. EMSCs can help gather macrophages in early stage to facilitate fat transplantation. The EMSCs' effect of promoting fat transplantation is lost after macrophages are removed with inhibitor, and the grafts become irregular tissue masses. In later stage, CCL2 expression is down-regulated in EMSCs-containing grafts, which is consistent with the decreased number of macrophages and decreased fibrosis. This stage-specific ability to regulate macrophage mobilization plays an important role in the process of promoting fat transplantation by EMSCs.

The cell line used for the human embryonic stem cells is not specifically defined, and all hESC cell lines can be used. In some embodiments, the human embryonic stem cell is selected from any cell line from the group consisting of Envy, CT3, CT1, CT2, CT4, H1, H7, H9, H13 and H14. All human induced pluripotent stem cell lines can also be used as it has been well known that they have the same ability as hESCs to differentiate to any cell types in the body including MSCs.

In some embodiments, the method for preparing mesenchymal stem cells includes steps of: differentiating human pluripotent stem cells into mesenchymal stem cells; and culturing the obtained mesenchymal stem cells by differentiation, and subculturing once every 5˜7 days.

The preparing method is the method for deriving and preparing human pluripotent stem cells into mesenchymal-like stem cells adopted in the patent with the filing No. CN201380036985.7.

An example of the present disclosure further provides use of mesenchymal stem cells in preparation of a formulation for activating a CCL2 signal pathway in early stage of fat transplantation, wherein the mesenchymal stem cells are derived from human pluripotent stem cells. It should be noted that the method for preparing mesenchymal stem cells in the present embodiment and subsequent embodiments is the same as the method for preparing mesenchymal stem cells in any of the above embodiments, and will not be repeated again.

An embodiment of the present disclosure further provides use of mesenchymal stem cells in preparation of a formulation for gathering macrophages in early stage of fat transplantation, wherein the mesenchymal stem cells are derived from human pluripotent stem cells.

An embodiment of the present disclosure further provides use of mesenchymal stem cells in preparation of a medicine for promoting regathering of adipose cells, wherein the mesenchymal stem cells are derived from human pluripotent stem cells.

An embodiment of the present disclosure further provides use of mesenchymal stem cells in preparation of a medicine for promoting revascularization, wherein the mesenchymal stem cells are derived from human pluripotent stem cells.

An embodiment of the present disclosure further provides use of mesenchymal stem cells in preparation of a medicine for improving survival rate of adipose cells, wherein the mesenchymal stem cells are derived from human pluripotent stem cells.

An embodiment of the present disclosure further provides use of mesenchymal stem cells in preparation of a medicine for inhibiting tissue fibrosis, wherein the mesenchymal stem cells are derived from human pluripotent stem cells.

In one or more embodiments, the tissue fibrosis is adipose tissue fibrosis.

In one or more embodiments, the subject is a subject receiving fat transplantation.

The features and performances of the present disclosure are further described in detail below in combination with examples.

### EXAMPLES

The present example provides use of EMSCs in fat transplantation.

Materials and Method

hESCs and EMSCs

All of the experiments in the present research were carried out following the guideline of US National Institutes of Health for human stem cell researches. All of the experimental schemes in the present research had been approved by Research Ethics Committee of University of Macau. Two hESCs cell lines were used, namely, Envy (GFP+) (Costa et al. 2005) and CT3 (Martins-Taylor, et al. 2011). The hESCs were differentiated into MSCs (EMSCs) through intermediate state of trophoblast cells (Wang et al. 2016).

Differentiation solution: EMSCs were cultured in α-MEM medium containing 20% fetal bovine serum, 1% non-essential amino acid, and 1% glutamine, under culture condition of 37° C. and 5% CO₂, while subculturing once every 5-7 days, and the generation number of EMSCs used in the example was from 6 to 9.

Human Fat Extract and Stromal Vascular Fraction (SVF)

Human fat extract was obtained from an orthopedic hospital, with corresponding human ethics scheme No. #BSERE17-APP019-FHS proved by University of Macau. Fat donors were healthy women of 30-60 years, and fat was extracted from their abdomens or thighs acting as filling tissues when they went to orthopedic hospital to receive plastic surgery. After signing the informed consent, a portion of excess fat extract was donated to research of the present project. According to reported experimental procedure (Moscatello et al. 2005), the fat extract was separated from liquid, oil and cell debris by centrifugation at 1500 rpm for 5 minutes. Subsequently, the fat extract was mixed with an equal volume of DMEM medium containing 10% fetal bovine serum and 10% dimethyl sulfoxide, and then the mixture was frozen at −80° C. in a volume of 10 ml per tube. The mixture was frozen for at most 2˜3 weeks before transplantation experiment.

In addition, SVF was isolated from fresh fat extract according to published experimental scheme (Fu et al. 2013). Briefly, the fat extract was digested with 0.075% collagenase I on a shaker at 150 rpm and 37° C. for 30 minutes. Subsequently, the resultant was centrifuged at 600 g for 10 minutes so as to remove mature adipose cells and connective tissues. Finally, cell pellet containing SVF was resuspended using PBS, and the cells in SVF were counted after staining by trypan blue.

Transplantation of Human Fat into Mice

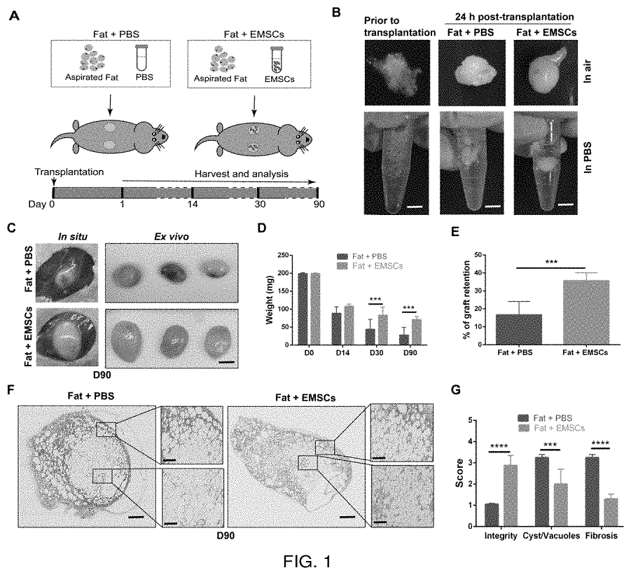

All of the animals were used following the Animal Use Guidelines of University of Macau and the experimental scheme approved by Animal Ethics Committee of University of Macau (#UMARE-043-2017). In the examples, athymic female nude mice of 17-20 g were taken as experimental subjects for human fat transplantation, with 4 mice in each group. Mice were fed in cages according to a strict process. 0.2 ml (200 mg) of human fat extract was mixed with 10⁶ EMSCs (Envy hESC cell line, GFP+) suspended in 20 μl of PBS acting as an experimental group (EMSC group); and the fat extract was mixed with 20 μl of PBS acting as a negative control group (PBS group). Refer to A in FIG. 1 for transplantation diagram.

According to a reported experimental scheme (Chung et al. 2013), the fat mixture of the experimental group or the control group was subcutaneously injected on two sides of the back of the mice using 1 ml syringe with 16 G needle. First, a subcutaneous channel was made with the needle under the skin of the back of the mice, and subsequently the fat mixture was injected into this channel. After injection, the skin wound was stitched with a 6/0 nylon wire, and no bleeding was seen after surgery. Animals were observed every day, and sampling and analysis were carried out at specific time points.

It should be noted that all of the transplantation mentioned in Test Examples 1˜5 were carried out using the method provided in the present example.

### Test Example 1

EMSCs Promote Fat Regathering and Retention

As shown in A in FIG. 1, after transplantation, mice were observed every day, and grafts were collected and analyzed at days 1, 14, 30 and 90.

Specifically, before transplantation, the extracted adipose tissues were structurally loose and could be dispersed in PBS. 24 h after transplantation, schematic diagrams of the grafts were as shown in B in FIG. 1, and it can be seen from the drawing that the adipose tissues in the PBS group were quickly separated into small masses in the PBS, while the adipose tissues in the EMSC group maintained an intact massive morphology in the PBS.

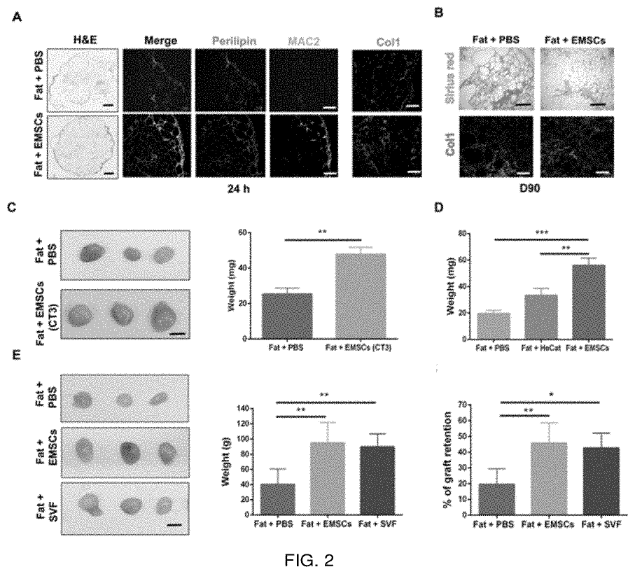

Immunostaining shows (FIG. 2) that the EMSC group contained more live adipose cells (perilipin⁺) and extracellular matrix (ECM) (collagen I positive, CoII⁺), the grafts in the EMSC group had clear edges and were rich in macrophages (MAC2⁺) (A in FIG. 2). Existing researches have shown that adipose cells and their ECM are severely damaged in extracted adipose tissues (Erdim et al. 2009; Eto et al. 2009). Results in FIG. 2 indicate that EMSCs may promote ECM remodeling and adipose cell regathering.

Refer to C in FIG. 1 for results at day 90 after transplantation, the grafts in the EMSC group were larger than those in the control group whether viewed in situ or compared ex vivo; in addition, viewing from the surface of isolated grafts, the control group had more dark areas (fibrosis), while the EMSC group had more red areas (capillary enrichment).

Meanwhile, the grafts in the EMSC group collected from different times had higher average weight than the control group (D in FIG. 1). For example, the average weight of the grafts in the control group and the EMSC group is 30 mg and 70 mg at day 90, respectively, 16.7% and 35.7% of the original weight, respectively (E in FIG. 1). Existing researches have shown that after fat transplantation, mechanical injury and insufficient blood supply may cause death of most transplanted adipose cells, reflected in damaged tissue integrity, and formation of vesicle cavities and fibrosis at the transplanted adipose cells, particularly in central positions (Kato, Mineda, et al. 2014).

Refer to F in FIG. 1 for H&E staining results of graft sections at day 90 (D90) after co-transplantation with PBS or EMSCs, refer to G in FIG. 1 for scores of graft integrity, number of cavities and fibrosis level obtained based on H&E staining of a plurality of D90 graft sections of the two groups, and it can be seen that compared with the control group, the grafts in the EMSC group contained more uniformly distributed and intact adipose cells and fewer cavities at day 90. Moreover, sirius red and collagen I staining showed that the control group contained more fibrosis tissues.

Similar results were also found in EMSCs derived from another hESC cell line (CT3) (Martins-Taylor, et al. 2011) (C in FIG. 2). However, compared with the PBS control group, HaCat cells (keratinocyte cell line, Boukamp et al. 1988) could not increase the weight of fat grafts (D in FIG. 2), demonstrating that EMSCs could specifically help the retention of fat grafts in vivo. In addition, effects of the EMSC and SVF isolated from the same fat extract were also compared, and we found that the effects of the two were similar, and the weight of the grafts at day 90 was greater than that of the control group (E in FIG. 2). These results indicate that EMSC and SVF are equally matched in the effect of promoting fat transplantation.

### Test Example 2

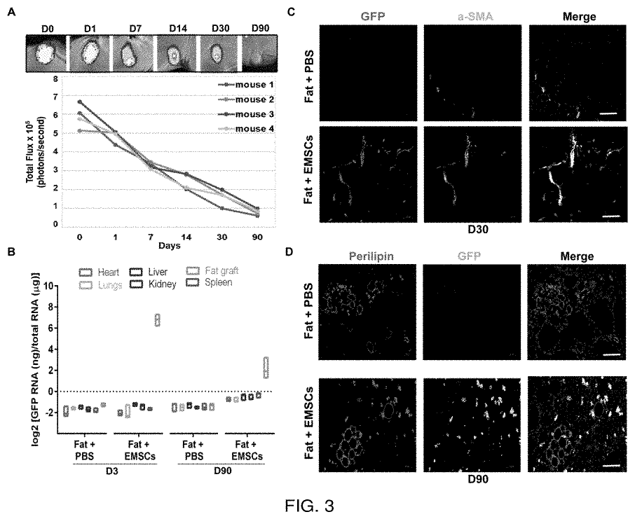

Survival of EMSCs in Fat Grafts (FIG. 3)

In order to track retention of live EMSCs in fat grafts, human fat extract was co-transplanted with EMSCs labeled with lentiviral vector luciferase (Wang, Kimbrel et al. 2014) into nude mice, and fluorescence signals of transplant sites were continuously tracked within 90 days after transplantation, refer to A in FIG. 3 for detection results.

As can be seen from A in FIG. 3, the fluorescence signals were detected to be gradually and significantly decreased after the transplantation, especially from day 0 (D0) to day 7 (D7), the signals sharply decreased (about 44%), and the fluorescence signals were almost undetected at day 90 (D90). No signal was detected in the PBS control group throughout the process (data is not given).

Tumorigenicity of EMSCs

After co-transplantation of GFP-positive EMSCs with human fat extract, fat grafts and major organs, including lung, liver, heart, kidney, spleen and so on, were collected from mice from day 3 to day 90, respectively. No tumor was observed in these tissues and organs based on appearance observation and histological analysis.

At D3 and D90 after transplantation, the total RNA was extracted after the isolated fat grafts were collected, and the RNA was reverse-transcribed into cDNA using High-Capacity cDNA Reverse Transcription Kit (Applied Biosystems) after quantification of the total RNA using NanoDrop. Quantitative PCR analysis was then performed on CFX96 Touch RT-PCR Instrument (Bio-Rad) using iTaq Universal SYBR Green Reagent (Bio-Rad). A standard curve was made with GFP-positive EMSCs as positive control, and different numbers of EMSCs and their resulting Ct values, so as to quantify the number of EMSCs that may exist in the grafts, refer to B in FIG. 3 for quantitative results.

As can be seen from the results, the RNA level of the GFP gene in the fat grafts drop sharply over time, while no RNA thereof is detected in other organs. These data show rapid disappearance of EMSCs in the grafts, indicating that EMSCs are unlikely to cause tumors.

Differentiation of EMSCs in Fat Grafts

At day 30 after transplantation, fat+ EMSCs (GFP⁺) group and fat+ PBS group were immunostained, respectively. GFP was stained into red, marker protein of smooth muscle cells, αSMA, was stained into green, and cell nuclei were counterstained with DAPI. Scale was 50 μm.

At day 90 after transplantation, fat+ EMSCs (GFP⁺) group and fat+ PBS group were immunostained, respectively. GFP was stained into green, perilipin was stained into red, and cell nuclei were counterstained with DAPI. Scale was 50 μm.

Immunostaining: paraffin sections were dewaxed and hydrated according to the following procedure: 100% xylene, 20 minutes; 100%, 100%, 95%, 95%, 90% and 80% ethanol, 5 minutes each, and finally washing the sections 3 times in PBS. Antigen repair was then performed in a citrate buffer, and the resultant was treated with 10% hydrogen peroxide for 10 minutes to remove endogenous peroxidase. The sections were further permeabilized with 0.3% Triton X-100 for 10 min, and sealed with 5% BSA for 1 hour after being washed with PBS 3 times. Subsequently, the treated sections were incubated using a specific primary antibody (including guinea pig anti-perilipin (Progen Biotechnik, Heidelberg, Germany), mouse anti-αSMA (eBiosciences, Sandiego, CA, USA), rat anti-mouse MAC-2 (Cedarlane, Burlington, Ontario, Canada), and goat anti-GFP (Abcam, Cambridge, United Kingdom)), and after further incubation using corresponding secondary antibody (including Alex Fluor 568-goat anti-guinea pig, Alex Fluor 488-goat anti-mouse, Alex Fluor 488-donkey anti-goat and Alex Fluor 594-donkey anti-goat antibodies (Invitrogen, Carlsbad, Calif.)), cell nuclei were counterstained with DAPI. All of the sections were photographed under a Carl Zeiss Axio Observer microscope and analyzed using ZEN imaging software.

Refer to C in FIG. 3 for immunostaining results of the grafts after D30, and refer to D in FIG. 3 for immunostaining results of the grafts after D90. Compared with the control group, at day 30 after transplantation, the grafts in the EMSC group contained more vascular cells (αSMA positive) and live adipose cells (perilipin positive). A part of these cells simultaneously showed GFP positivity, and these results indicate that a part of the EMSCs were also differentiated into vascular smooth muscle cells and adipose cells.

Host Mouse Tissues Participate in Transplantation of Human Fat

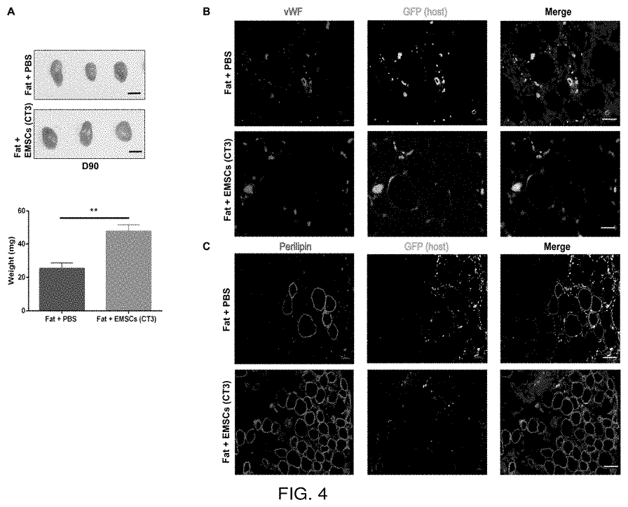

GFP mice were used as subjects for human fat transplantation. The fat extract was mixed with CT3 hESC-drived EMSCs and the mixture was injected subcutaneously to two sides of the back of GFP mice. Similar results were observed as in nude mice, and compared with the control group, the grafts in the EMSC group were larger, heavier at day 90 after transplantation (A in FIG. 4).

Besides, in addition to a small amount of cells showing von Willebrand factor (VWF, marker gene of vascular endothelial cells) and GFP double positivity (B in FIG. 4), all of the perilipin-positive cells were not GFP-positive (C in FIG. 4), and this result indicates that the host cell contributes very little to fat regeneration and revascularization.

### Test Example 3

Transcriptomic Analysis of Fat Grafts

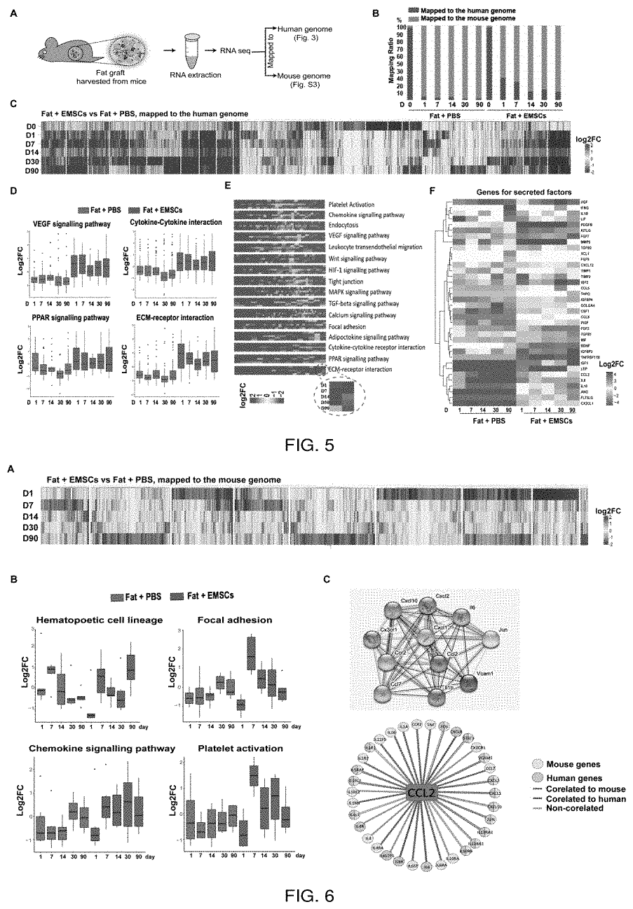

In order to further illustrate the composition of the grafts and reveal the molecular mechanism of the EMSC promoting fat transplantation, RNA sequencing analysis was performed on the fat grafts in the EMSC group and the PBS group at different time points (at days 1, 7, 14, 30 and 90), and refer to A in FIG. 5 for schematic view of sample separation and analysis.

RNA sequencing: 4 fat grafts in the same group at the same time point were mixed together, and the grafts were lysed in Trizol (Thermofisher) using a tissue homogenizer. Subsequently, RNA extraction was performed using RNeasy Lipid Tissue Mini Kit (Qiagen), and quantitative and qualitative evaluation was carried out for RNA using Nano RNA Bioanalyzer (Agilent Technologies). The next generation of sequencing library was prepared using samples with RNA integrity number (RIN) of 8 or greater by NEBNext Ultra TM Directional RNA Library Prep Kit for Illumina (New England BioLabs).

Fat before transplantation and fat mixed with EMSCs were used as standardized controls. Subsequently, sequencing reading values were matched to human and mouse genomes, respectively, and in order to prevent confusion, values that simultaneously match human and mouse genomes are excluded.

Refer to B in FIG. 5 for proportions of RNA sequencing results of the fat+ PBS group and fat+ EMSC group before transplantation and at D1-D90 after transplantation mapped to human genomes (blue) and mouse genomes (orange).

As can be seen from the results, 24 hours after transplantation, the proportion of human RNA in the control group in the total RNA was decreased from 98% to 5%, while the mouse RNA was increased from 2% to 98%, which continued until day 90 after the transplantation. The human RNA in the EMSC group was gradually reduced from 30% at 24 hours to 12% at day 90. Since lipid droplets occupy most of the space in adipose cells, the amount of RNA in fat extract alone is much less than in fat grafts mixed with EMSC or impregnated by host cells. Thus, the decrease in human RNA proportion and the increase in mouse RNA proportion may be construed as rapid death of a large proportion of human adipose cells after transplantation, and impregnation of a large number of mouse cells into the grafts in the meanwhile. This conclusion is consistent with the previously observed rapid contraction of the fat grafts after transplantation. Reduction of graft contraction by EMSCs may be associated with increase of graft cell number and improvement of survival rate of adipose cells in the grafts.

After matching the sequencing values of the EMSC group and the PBS group at each time point after transplantation to the human genomes, many differentially expressed genes (DEGs) were found, and refer to C in FIG. 5 for expression heatmap of human genes in RNA sequencing of samples. In a color scale, colors were assigned from blue to red (from bottom to top) according to Log2 fold change of expression (FC) (−2 to 2), respectively representing descending or rising of corresponding gene expression level in the EMSCs-containing transplantation group with respect to the PBS control group at each time point.

Refer to D in FIG. 5 for box diagram of expression changes of human genes related to fat regeneration in the EMSCs transplantation group, and each graph represents one signal pathway or functional group. According to gene ontology annotation, refer to E in FIG. 5 for a heatmap drawn according to significantly enriched DEGs in the samples; and the color display manner is consistent with that in C in FIG. 5. As can be seen from D and E in FIG. 5, many up-regulated genes were associated with VEGF pathway, interaction between cytokines, PPAR signal pathway and ECM receptor interaction, which is consistent with the phenomena of revascularization, fat regeneration and ECM remodeling of grafts in the EMSC group.

In agreement with this, expression levels of secreted human chemokines and cytokines including CCL2, CCLS, CCL8 and IL16 were higher in the EMSC group, compared with the control group, specifically referring to F in FIG. 5, a heatmap of genes encoding human excreted factors in the samples, in which levels of gene up-regulation and down-regulation in the post-transplantation samples in each group were compared with those in corresponding samples before transplantation (D0) (A in FIG. 6). On the other hand, the mouse immune response and the expression of inflammation-related genes including target genes in CCL2 signal pathway was also higher in the EMSC group (B in FIG. 6).

CCL2 (or MCP1) is a cytokine with high affinity for its receptor CCR2, which is consistent with the finding that the mouse Ccr2 receptor was significantly associated with human CCL2 by Pearson association analysis (C in FIG. 6), besides, CCL2 is also a chemokine for monocytes and macrophages. Macrophages play different roles in fat transplantation in different stages, including promoting ECM remodeling and angiogenesis in early stage and promoting fibrosis in later stage.

In addition, RNA sequencing results also show that the gene expressions of the two are correlated, which indicates that CCL2 secreted by EMSCs might gather macrophages to arrive at the site of the grafts by binding and activating Ccr2 on mouse macrophages.

### Test Example 4

The EMSC's Effect of Promoting Fat Transplantation Depends on CCL2-Ccr2 Signal Pathway.

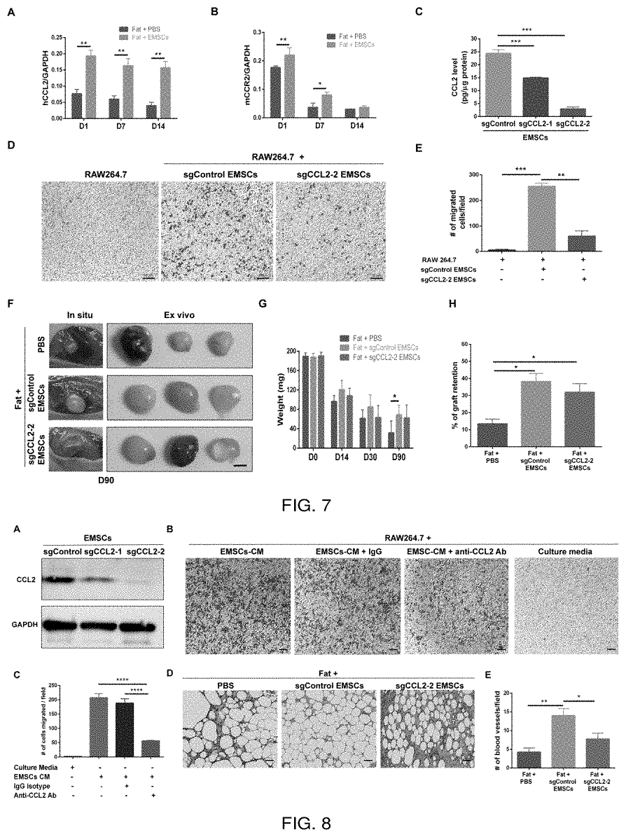

Expression of human CCL2 and mouse Ccr2 in fat grafts in early stage (days 1, 7 and 14) of transplantation was confirmed by qPCR, wherein refer to A in FIG. 7 for RNA expression levels of CCL2, refer to B in FIG. 7 for RNA expression levels of Ccr2, and GAPDH is used as an internal reference in A and B in FIG. 7,*P<0.05, “P<0.01. The results show that, although the expression of Ccr2 decreased in both of the two groups at day 7 and day 14, the expression of CCL2 and Ccr2 was higher in the EMSC group than in the control group.

Subsequently, the CCL2 gene in EMSC was knocked out using lentivirus-mediated CCL2-specific sgRNA/Cas9 (EMSC sgCCL2-1 and EMSC sgCCL2-2). Meanwhile, lentivirus-mediated out-of-order sgRNA/Cas 9-transduced EMSC (EMSC sgControl) was taken as negative control.

Knockout of CCL2 gene: in order to construct a lentiviral vector for sgCCL2, a CCL2-specific oligonucleotide sequence was designed and synthesized and cloned into BsmBl-digested LentiCRISPRv2 (Addgene #52961). Oligonucleotide sequences without recognition sites in the human genome were designed as negative control. The constructed lentiviral shuttle vector will then be co-transfected into 293FT cells using lipofectamine 3000 (invitrogen) with envelope plasmid pMD2.G, packaging plasmid pCMV delta R8.2 (Addgene #12259 and 12263) so as to package virus. The collected viruses were then infected to EMSCs, and the resultant was screened using puromycin (1 μg/ml) for 2 weeks. Finally, knockout verification was performed for the obtained EMSCs. To verify the knockout of CCL2, lysates of EMSC were collected and the expression of CCL2 was detected using Western Blot. First, the incubation was carried out overnight with CCL2 (Abcam, ab214819) antibody, followed by incubation with corresponding HRP-labeled secondary antibody. Finally, imaging was performed in ChemiDoc imaging system (Bio-Rad) using Clarity TM Western ECL substrate. In order to detect the secretion of CCL2, the supernatant obtained by culturing EMSCs for 48 hours was collected and then the secretion of CCL2 was measured using CCL2 ELISA kit (R&D systems, DCP00) according to the manufacturer's instruction.

Refer to A in FIG. 8 for a result of Western Blot, refer to C in FIG. 7 for the level of CCL2 in the culture supernatant of wild-type and CCL3-knockout EMSCs detected by an immunoenzyme-linked reaction (“P<0.001, ***p<0.0001), and it can be seen from the result that the expression of CCL2 was nearly halved in EMSC sgCCL2-1, and almost completely disappears in EMSC sgCCL2-2. Therefore, EMSC sgCCL2-2 was used for subsequent experiments.

Response of Macrophages to CCL2 Knockout and Non-knockout EMSC.

The ability of RAW264.7 (mouse macrophage cell line) to migrate towards EMSC cultured on bottom surface was tested by Transwell system.

Macrophage migration experiment by Transwell: 1×10⁵ EMSCs were inoculated onto the bottom surface of Transwell culture plate using a Transwell chamber system with a pore size of 3.0-μm, and after overnight culture, 1×10⁵ RAW264.7 macrophages were inoculated onto membranes of the Transwell chambers. After further incubation at 37° C. and 5% CO₂ for 24 hours, the Transwell chambers were collected, fixed and then stained with crystal violet dye, and then 5 regions were randomly selected under microscope to photograph (20×) and count transmembrane macrophages (“P<0.01, n=3).

Refer to D in FIG. 7 for photographing result, and refer to E in FIG. 7 for counting result. As can be seen from the results, fewer RAW264.7 cells migrated in the EMSC group with CCL2 knockout, compared with the control group.

Besides, the migration ability of RAW264.7 cells cultured in EMSC-conditioned medium treated with CCL2 neutralizing antibody also declined (B in FIG. 8 and C in FIG. 8). These results indicate that EMSCs may gather macrophages through the CCL2/Ccr2 signal pathway.

Effect of CCL2 in EMSC Promoting Fat Transplantation.

Human fat extract was co-transplanted with EMSC sgCCL2-2, EMSC sgControl and PBS, respectively, and samples were collected and weighed at days 14, 30, and 90 after transplantation.

Refer to F in FIG. 7 for photographing results of grafts in situ and ex vivo at D90 after transplantation, wherein the fat grafts in the EMSC sgCCL2-2 group contained more dark (fibrotic and necrotic) regions and vesicle cavities at day 90 than EMSC sgControl.

Refer to G in FIG. 7 for weight results of grafts at different time points, and it can be seen that the fat grafts in each group gradually shrank and lightened in weight from day 0 to day 90. The weight of the grafts was relatively greater in the EMSC sgControl group than in the PBS group. The weight of the grafts in the EMSC sgCCL2-2 group was only slightly and insignificantly reduced, compared with the EMSC sgControl group.

Refer to H in FIG. 7 for results of retention rate of grafts at D90 compared with grafts at D0 (*P<0.05, n=4). The grafts in the EMSC sgControl group contained more intact adipose cells and vessels, and less fibrosis, necrosis and fewer vesicle cavities than the PBS and EMSC sgCCL2-2 groups (see D and E in FIG. 8).

These results indicate that at least part of the mechanism by which EMSCs promote fat transplantation is accomplished by secreting CCL2 to gather macrophages.

### Test Example 5

EMSCs Mobilize Macrophages in Different Stages of Fat Transplantation.

As the inventors found that early EMSC-fat grafts contained significantly increased macrophages, the present test example demonstrated how these macrophages functioned in the fat grafts.

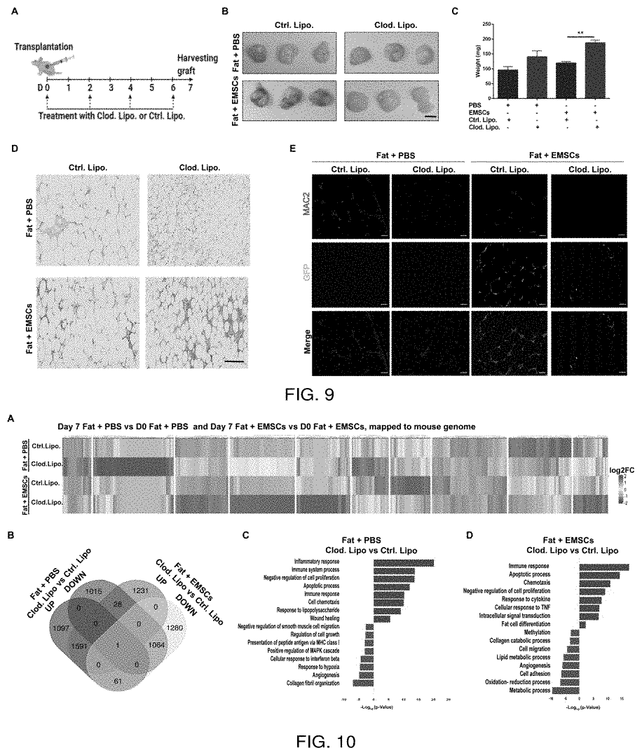

Refer to A in FIG. 9 for schematic view of the experiment. In order to clear away macrophages, clodronate liposomes and PBS liposomes (Liposoma BV) were respectively injected subcutaneously into the fat grafts every two days from the start of fat transplantation, at days 0, 2, 4 and 6 after transplantation, respectively, by 0.5 ml/100 g of animal body weight, and then graft samples were collected at day 7.

Refer to B in FIG. 9 for diagram of the grafts at day 7 after transplantation. As can be seen from the results, when injected with the PBS liposomes (negative control), there are more capillaries on the surface of the grafts (red surface) in the EMSCs group, compared with the PBS control group (white surface). When injected with the clodronate liposomes, the surface of the grafts appeared white in both the PBS control group and the EMSCs group, indicating no presence of capillaries.

Refer to C in FIG. 9 for weight results of the grafts at day 7 after transplantation, and it can be seen that all of the grafts treated with the clodronate liposomes were heavier in weight than those treated with the PBS liposomes in both the PBS control group and the EMSCs group.

In addition, macrophage clearance and EMSC (GFP positive) retention in the grafts were also confirmed by MAC2 and GFP immunostaining, respectively. Refer to D in FIG. 9 for H&E staining results after the grafts were sectioned at day 7 after transplantation, and refer to E in FIG. 9 for results of graft immunofluorescence staining at day 7 after transplantation. It is found that in the grafts at day 7 treated with clodronate liposomes, adipose cells were replaced by random cell clusters.

The grouping of differentially expressed genes was obtained by analyzing RNA sequencing results of different groups and is shown by means of heatmap (A in FIG. 10). In addition, Venn diagram shows that up-regulated expression of 1591 genes and down-regulated expression of 1064 genes were found in both the control group and the EMSC group (B in FIG. 10). Functional and signal pathway analysis shows that compared with the group treated with the PBS liposomes, the gene expression associated with immunity, inflammatory response and apoptosis is up-regulated, while the gene expression associated with cell adhesion and angiogenesis is down-regulated in the EMSC fat grafts treated with the clodronate liposomes (C in FIG. 10). Similar expression profile changes also occurred in the PBS fat grafts (D in FIG. 10). These results show that regardless of the presence or absence of EMSCs, macrophages are essential to fat transplantation, and they function by inhibiting inflammation and apoptosis, promoting cell adhesion and revascularization. The EMSC's effect of promoting transplantation may be accomplished partially by gathering and activating macrophages in early stage.

Since the aggregated macrophages will promote fibrosis in later stage of fat transplantation, reducing aggregation of macrophages in this stage can help remodeling of fat grafts. Interestingly, our results show that at day 90 of transplantation, the EMSC group contained fewer macrophages than the PBS group, which was consistent with the fact that it contained less fibrosis (E in FIG. 9). These results indicate that EMSC plays different roles on macrophage mobilization in different stages during fat transplantation, for example, gathering macrophages in early stage of transplantation to promote graft survival, while clearing away macrophages in later stage to reduce effect of fibrosis.

The examples described above are only some examples of the present disclosure, rather than all examples. The detailed description of the examples of the present disclosure is not intended to limit the scope of the present disclosure as claimed, but merely represents chosen examples of the present disclosure. Based on the examples of the present disclosure, all of other examples obtained by those ordinarily skilled in the art without any inventive effort shall fall within the scope of protection of the present disclosure.

### INDUSTRIAL APPLICABILITY

The examples of the present disclosure provide use of mesenchymal stem cells in preparation of a formulation for promoting fat transplantation, wherein the mesenchymal stem cells are derived from human pluripotent stem cells (abbreviated as EMSCs). Research finds that compared with MSCs derived from somatic cells, EMSCs are more stable in quality, are not affected by donor's physical quality, disease and treatment process, and can promote fat transplantation by enhancing tissue remodeling, angiogenesis, adipose cell survival and decreasing tissue fibrosis.

What is claimed is:

1. Method for promoting fat transplantation, comprising applying mesenchymal stem cells to a subject in need thereof, wherein the mesenchymal stem cells are derived from human pluripotent stem cells including human embryonic stem cells and induced pluripotent stem cells.

2. The method for promoting fat transplantation according to claim 1, wherein the method comprises activating a CCL2 signal pathway in an early stage of fat transplantation, comprising applying the mesenchymal stem cells to a subject in need thereof.

3. The method for promoting fat transplantation according to claim 1, wherein the method comprises gathering macrophages in an early stage of fat transplantation, comprising applying the mesenchymal stem cells to a subject in need thereof.

4. The method for promoting fat transplantation according to claim 1, wherein the method comprises promoting regathering of adipose cells, comprising applying mesenchymal stem cells to a subject in need thereof.

5. The method for promoting fat transplantation according to claim 1, wherein the method comprises promoting revascularization, comprising applying mesenchymal stem cells to a subject in need thereof.

6. The method for promoting fat transplantation according to claim 1, wherein the method comprises improving survival rate of adipose cells, comprising applying mesenchymal stem cells to a subject in need thereof.

7. The method for promoting fat transplantation according to claim 1, wherein the method comprises inhibiting tissue fibrosis, comprising applying mesenchymal stem cells to a subject in need thereof.

8. The method according to claim 7, wherein the tissue fibrosis is adipose tissue fibrosis.

9. The method according to claim 1, wherein the subject is a subject receiving fat transplantation.

10. The method according to claim 1, wherein a method for preparing the mesenchymal stem cells comprises: differentiating the human pluripotent stem cells into the mesenchymal stem cells, culturing differentiated mesenchymal stem cells, and subculturing once every 5-7 days.

11. The method according to claim 1, wherein the human pluripotent stem cells are selected from any one cell line which is selected from the group consisting of Envy, CT3, CT1, CT2, CT4, H1, H7, H9, H13 and H14.

12. The method according to claim 2, wherein the human embryonic stem cells are selected from any one cell line which is selected from the group consisting of Envy, CT3, CT1, CT2, CT4, H1, H7, H9, H13 and H14.

13. The method according to claim 3, wherein the human embryonic stem cells are selected from any one cell line which is selected from the group consisting of Envy, CT3, CT1, CT2, CT4, H1, H7, H9, H13 and H14.

14. The method according to claim 4, wherein the human embryonic stem cells are selected from any one cell line which is selected from the group consisting of Envy, CT3, CT1, CT2, CT4, H1, H7, H9, H13 and H14.

15. The method according to claim 5, wherein the human embryonic stem cells are selected from any one cell line which is selected from the group consisting of Envy, CT3, CT1, CT2, CT4, H1, H7, H9, H13 and H14.

16. The method according to claim 6, wherein the human embryonic stem cells are selected from any one cell line which is selected from the group consisting of Envy, CT3, CT1, CT2, CT4, H1, H7, H9, H13 and H14.

17. The method according to claim 7, wherein the human embryonic stem cells are selected from any one cell line which is selected from the group consisting of Envy, CT3, CT1, CT2, CT4, H1, H7, H9, H13 and H14.

18. The method according to claim 8, wherein the human embryonic stem cells are selected from any one cell line which is selected from the group consisting of Envy, CT3, CT1, CT2, CT4, H1, H7, H9, H13 and H14.

19. The method according to claim 9, wherein the human embryonic stem cells are selected from any one cell line which is selected from the group consisting of Envy, CT3, CT1, CT2, CT4, H1, H7, H9, H13 and H14.

20. The method according to claim 10, wherein the human embryonic stem cells are selected from any one cell line which is selected from the group consisting of Envy, CT3, CT1, CT2, CT4, H1, H7, H9, H13 and H14.
